(19) United States
(12) Patent Application Publication
(10) Pub. No.: US 2007/0218530 A1
(43) Pub. Date: Sep. 20, 2007

(54) METHODS FOR ENZYMATIC HYDROLYSIS OF LIGNOCELLULOSE

(75) Inventors: Nicholas B. Duck, Apex, NC (US); Brian Carr, Raleigh, NC (US); Michael G. Koziel, Raleigh, NC (US); Nadine Carozzi, Raleigh, NC (US); Brian Vande Berg, Durham, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Athenix Corporation

(21) Appl. No.: 11/748,727

(22) Filed: May 15, 2007

Related U.S. Application Data
(63) Continuation of application No. 10/426,111, filed on Apr. 29, 2003.
(60) Provisional application No. 60/376,527, filed on Apr. 30, 2002.
(60) Provisional application No. 60/432,750, filed on Dec. 12, 2002.

(51) Int. Cl. C12P 19/04; C12P 19/02; C12P 19/12

(52) U.S. Cl. 435/101; 435/100; 435/105

(57) ABSTRACT

Compositions and methods for biomass conversion are provided. Compositions comprise novel enzyme mixtures that can be used directly on lignocellulose substrate. Methods involve converting lignocellulosic biomass to free sugars and small oligosaccharides with enzymes that break down lignocellulose. Novel combinations of enzymes are provided that provide a synergistic release of sugars from plant biomass. Also provided are methods to identify enzymes, strains producing enzymes, or genes that encode enzymes capable of degrading lignocellulosic material to generate sugars.

### CROSS REFERENCE TO RELATED APPLICATION

This application claims the benefit of U.S. application Ser. No. 10/426,111, filed Apr. 29, 2003, U.S. Provisional Application Ser. No. 60/376,527, filed Apr. 30, 2002, and U.S. Provisional Application Ser. No. 60/432,750, filed Dec. 12, 2002, the contents of which are herein incorporated by reference in their entirety.

### FIELD OF THE INVENTION

Methods to produce free sugars and oligosaccharides from plant material are provided.

### BACKGROUND OF THE INVENTION

Carbohydrates constitute the most abundant organic compounds on earth. However, much of this carbohydrate is sequestered in complex polymers including starch (the principle storage carbohydrate in seeds and grain), and a collection of carbohydrates and lignin known as lignocellulose. The main carbohydrate components of lignocellulose are cellulose, hemicellulose, and glucans. These complex polymers are often referred to collectively as lignocellulose.

Starch is a highly branched polysaccharide of alpha-linked glucose units, attached by alpha-1,4 linkages to form linear chains, and by alpha-1,6 bonds to form branches of linear chains. Cellulose, in contrast, is a linear polysaccharide composed of glucose residues linked by beta-1,4 bonds. The linear nature of the cellulose fibers, as well as the stoichiometry of the beta-linked glucose (relative to alpha) generates structures more prone to interstrand hydrogen bonding than the highly branched alpha-linked structures of starch. Thus, cellulose polymers are generally less soluble, and form more tightly bound fibers than the fibers found in starch.

Hemicellulose is a complex polymer, and its composition often varies widely from organism to organism, and from one tissue type to another. In general, a main component of hemicellulose is beta-1,4-linked xylose, a five carbon sugar. However, this xylose is often branched as beta-1,3 linkages, and can be substituted with linkages to arabinose, galactose, mannose, glucuronic acid, or by esterification to acetic acid. Hemicellulose can also contain glucan, which is a general term for beta-linked six carbon sugars.

The composition, nature of substitution, and degree of branching of hemicellulose is very different in dicot plants as compared to monocot plants. In dicots, hemicellulose is comprised mainly of xyloglucans that are 1,4-beta-linked glucose chains with 1,6-beta-linked xylosyl side chains. In monocots, including most grain crops, the principle components of hemicellulose are heteroxylans. These are primarily comprised of 1,4-beta-linked xylose backbone polymers with 1,3-beta linkages to arabinose, galactose and mannose as well as xylose modified by ester-linked acetic acids. Also present are branched beta glucans comprised of 1,3- and 1,4-beta-linked glucosyl chains. In monocots, cellulose, heteroxylans and beta glucans are present in roughly equal amounts, each comprising about 15-25% of the dry matter of cell walls.

The sequestration of such large amounts of carbohydrates in plant biomass provides a plentiful source of potential energy in the form of sugars, both five carbon and six carbon sugars that could be utilized for numerous industrial and agricultural processes. However, the enormous energy potential of these carbohydrates is currently under-utilized because the sugars are locked in complex polymers, and hence are not readily accessible for fermentation. Methods that generate sugars from plant biomass would provide plentiful, economically-competitive feedstocks for fermentation into chemicals, plastics, and fuels.

Current processes to generate soluble sugars from lignocellulose are complex. A key step in the process is referred to as pretreatment. The aim of pretreatment is to increase the accessibility of cellulose to cellulose-degrading enzymes, such as the cellulase mixture derived from fermentation of the fungus Trichoderma reesei. Current pretreatment processes involve steeping lignocellulosic material such as corn stover in strong acids or bases under high temperatures and pressures. Such chemical pretreatments degrade hemicellulose and/or lignin components of lignocellulose to expose cellulose, but also create unwanted by-products such as acetic acid, furfural, hydroxymethyl furfural and gypsum. These products must be removed in additional processes to allow subsequent degradation of cellulose with enzymes or by a co-fermentation process known as simultaneous saccharification and fermentation (SSF).

The conditions currently used for chemical pretreatments require expensive reaction vessels, and are energy intensive. Chemical pretreatment occurring at high temperatures and extreme pH conditions (for example 160° C. and 1.1% sulfuric acid at 12 atm. pressure) are not compatible with known cellulose-degrading enzymes. Further, these reactions produce compounds that must be removed before fermentation can proceed. As a result, chemical pretreatment processes currently occur in separate reaction vessels from cellulose degradation, and must occur prior to cellulose degradation.

Thus, methods that are more compatible with the cellulose degradation process, do not require high temperatures and pressures, do not generate toxic waste products, and require less energy, are desirable.

For these reasons, efficient methods are needed for biomass conversion.

### SUMMARY OF INVENTION

Methods for generating free sugars and oligosaccharides from lignocellulosic biomass are provided. These methods involve converting lignocellulosic biomass to free sugars and small oligosaccharides with enzymes that break down lignocellulose. Enzymes used in the conversion process can degrade any component of lignocellulose and include but are not limited to: cellulases, xylanases, ligninases, amylases, proteases, lipidases and glucuronidases. The enzymes of the invention can be provided by a variety of sources. That is, the enzymes may be bought from a commercial source. Alternatively, the enzymes can be produced recombinantly, such as by expression either in microorganisms, fungi, i.e., yeast, or plants.

Novel combinations of enzymes are provided. The combinations provide a synergistic release of sugars from plant biomass. The synergism between enzyme classes requires less enzyme of each class and facilitates a more complete release of sugars from plant biomass, allowing more efficient conversion of biomass to simple sugars. Efficient biomass conversion will reduce the costs of sugars useful to generate products including specialty chemicals, chemical feedstocks, plastics, solvents and fuels by chemical conversion or fermentation.

Also provided are methods to identify enzymes, strains producing enzymes, or genes that encode enzymes capable of degrading lignocellulosic material to generate sugars. These methods involve assays based on degradation of lignocellulosic biomass and quantitation of the released sugar. Additionally, methods that utilize such assays to screen microbes, enzymes, or genes and quantify the ability of the enzyme to degrade lignocellulose are provided. These methods are useful in identifying proteins (enzymes) that are most useful for incorporation into biomass conversion methods described above.

Also provided are methods to identify the optimum ratios and compositions of enzymes with which to degrade each lignocellulosic material. These methods include tests to identify the optimum enzyme composition and ratios for efficient conversion of any lignocellulosic substrate to its constituent sugars.

Also provided are methods to identify novel enzymes, enzyme combinations or enzyme uses. These methods involve testing enzymes in assays utilizing hydrolyzed material remaining after enzymatic digestion as above. This method identifies enzymes that result in further hydrolysis of corn stover and other lignocellulosic materials, resulting in additional sugar release.

### DETAILED DESCRIPTION

Methods and compositions for the conversion of plant biomass to sugars and oligosaccharides that can be fermented or chemically converted to useful products are provided. That is, methods for degrading substrate using enzyme mixtures to liberate sugars are provided. Furthermore, methods to identify novel enzymes or strains producing enzymes or genes encoding enzymes useful in the method are described. The compositions of the invention include synergistic enzyme combinations that break down lignocellulose. Such enzyme combinations or mixtures synergistically degrade complex biomass to sugars and will generally include a cellulase with at least one auxiliary enzyme.

Enzyme Compositions

“Auxiliary enzyme”, “auxiliary enzymes”, “auxiliary enzyme mix”, “catalytic mixture” or “catalytic mix” are defined as any enzyme(s) that increase or enhance sugar release from biomass. This can include enzymes that when contacted with biomass in a reaction, increase the activity of subsequent enzymes (e.g. cellulases). Alternatively, the auxiliary enzyme(s) can be reacted in the same vessel as other enzymes (e.g. cellulase). While it is understood that many classes of enzymes may function as auxiliary enzymes, in particular auxiliary enzymes can be composed of (but not limited to) enzymes of the following classes: cellulases, xylanases, ligninases, amylases, proteases, lipidases and glucuronidases. Many of these enzymes are representatives of class EC 3.2.1, and thus other enzymes in this class may be useful in this invention. An auxiliary enzyme mix may be composed of enzymes from (1) commercial suppliers; (2) cloned genes expressing enzymes; (3) complex broth (such as that resulting from growth of a microbial strain in media, wherein the strains secrete proteins and enzymes into the media; (4) cell lysates of strains grown as in (3); and, (5) plant material expressing enzymes capable of degrading lignocellulose.

It is recognized that any combination of auxiliary enzymes may be utilized. The enzymes may be used alone or in mixtures including, but not limited to, at least a cellulase; at least a xylanase; at least a ligninase; at least an amylase; at least a protease; at least a lipidase; at least a glucuronidase; at least a cellulase and a xylanase; at least a cellulase and a ligninase; at least a cellulase and an amylase; at least a cellulase and a protease; at least a cellulase and a lipidase; at least a cellulase and a glucuronidase; at least a xylanase and a ligninase; at least a xylanase and an amylase; at least a xylanase and a protease; at least a xylanase and a lipidase; at least a xylanase and a glucuronidase; at least a ligninase and an amylase; at least a ligninase and a protease; at least a ligninase and a lipidase; at least a ligninase and a glucuronidase; at least an amylase and a protease; at least an amylase and a lipidase; at least an amylase and a glucuronidase; at least a protease and a lipidase; at least a protease and a glucuronidase; at least a lipidase and a glucuronidase; at least a cellulase, a xylanase and a ligninase; at least a xylanase, a ligninase and an amylase; at least a ligninase, an amylase and a protease; at least an amylase, a protease and a lipidase; at least a protease, a lipidase and a glucuronidase; at least a cellulase, a xylanase and an amylase; at least a cellulase, a xylanase and a protease; at least a cellulase, a xylanase and a lipidase; at least a cellulase, a xylanase and a glucuronidase; at least a cellulase, a ligninase and an amylase; at least a cellulase, a ligninase and a protease; at least a cellulase, a ligninase and a lipidase; at least a cellulase, a ligninase and a glucuronidase; at least a cellulase, an amylase and a protease; at least a cellulase, an amylase and a lipidase; at least a cellulase, an amylase and a glucuronidase; at least a cellulase, a protease and a lipidase; at least a cellulase, a protease and a glucuronidase; at least a cellulase, a lipidase and a glucuronidase; at least a cellulase, a xylanase, a ligninase and an amylase; at least a xylanase, a ligninase, an amylase and a protease; at least a ligninase, an amylase, a protease and a lipidase; at least an amylase, a protease, a lipidase and a glucuronidase; at least a cellulase, a xylanase, a ligninase and a protease; at least a cellulase, a xylanase, a ligninase and a lipidase; at least a cellulase, a xylanase, a ligninase and a glucuronidase; at least a cellulase, a xylanase, an amylase and a protease; at least a cellulase, a xylanase, an amylase and a lipidase; at least a cellulase, a xylanase, an amylase and a glucuronidase; at least a cellulase, a xylanase, a protease and a lipidase; at least a cellulase, a xylanase, a protease and a glucuronidase; at lease a cellulase, a xylanase, a lipidase and a glucuronidase; at least a cellulase, a ligninase, an amylase and a protease; at least a cellulase, a ligninase, an amylase and a lipidase; at least a cellulase, a ligninase, an amylase and a glucuronidase; at least a cellulase, a ligninase, a protease and a lipidase; at least a cellulase, a ligninase, a protease and a glucuronidase; at least a cellulase, a ligninase, a lipidase and a glucuronidase; at least a cellulase, an amylase, a protease and a lipidase; at least a cellulase, an amylase, a protease and a glucuronidase; at least a cellulase, an amylase, a lipidase and a glucuronidase; at least a cellulase, a protease, a lipidase and a glucuronidase; at least a cellulase, a xylanase, a ligninase, an amylase and a protease; at least a cellulase, a xylanase, a ligninase, an amylase and a lipidase; at least a cellulase, a xylanase, a ligninase, an amylase and a glucuronidase; at least a cellulase, a xylanase, a ligninase, a protease and a lipidase; at least a cellulase, a xylanase, a ligninase, a protease and a glucuronidase; at least a cellulase, a xylanase, a ligninase, a lipidase and a glucuronidase; at least a cellulase, a xylanase, an amylase, a protease and a lipidase; at least a cellulase, a xylanase, an amylase, a protease and a glucuronidase; at least a cellulase, a xylanase, an amylase, a lipidase and a glucuronidase; at least a cellulase, a xylanase, a protease, a lipidase and a glucuronidase; at least a cellulase, a ligninase, an amylase, a protease and a lipidase; at least a cellulase, a ligninase, an amylase, a protease and a glucuronidase; at least a cellulase, a ligninase, an amylase, a lipidase and a glucuronidase; at least a cellulase, a ligninase, a protease, a lipidase and a glucuronidase; at least a cellulase, an amylase, a protease, a lipidase and a glucuronidase; at least a xylanase, a ligninase, an amylase, a protease and a lipidase; at least a xylanase, a ligninase, an amylase, a protease and a glucuronidase; at least a xylanase, a ligninase, an amylase, a lipidase and a glucuronidase; at least a xylanase, a ligninase, a protease, a lipidase and a glucuronidase; at least a xylanase, an amylase, a protease, a lipidase and a glucuronidase; at least a ligninase, an amylase, a protease, a lipidase and a glucuronidase; at least a cellulase, a xylanase, a ligninase, an amylase, a protease, and a lipidase; at least a cellulase, a xylanase, a ligninase, an amylase, a protease and a glucuronidase; at least a cellulase, a xylanase, a ligninase, an amylase, a lipidase and a glucuronidase; at least a cellulase, a xylanase, a ligninase, a protease, a lipidase and a glucuronidase; at least a cellulase, a xylanase, an amylase, a protease, a lipidase and a glucuronidase; at least a cellulase a ligninase, an amylase, a protease, a lipidase, and a glucuronidase; at least a xylanase, a ligninase, an amylase, a protease, a lipidase and a glucuronidase; at least a cellulase, a xylanase, a ligninase, an amylase, a protease, a lipidase and a glucuronidase; and the like. It is understood that as described above, an auxiliary mix may be composed of a member of each of these enzyme classes, several members of one enzyme class (such as two or more xylanases), or any combination of members of these enzyme classes (such as a protease, an exocellulase, and an endoxylanase; or a ligninase, an exoxylanase, and a lipidase).

The auxiliary enzymes may be reacted with substrate or biomass in a pretreatment prior to the addition of cellulase, or alternatively, the cellulase may be included in any of the enzyme mixtures. That is, the cellulase may be added in any of the enzyme mixtures listed above. The enzymes may be added as a crude, semi-purified, or purified enzyme mixture. The temperature and pH of the substrate and enzyme combination may vary to increase the activity of the enzyme combinations. Likewise, the temperature and pH may be varied at the addition of one or more of the enzymes to increase activity of the enzyme. However, the pH and temperature adjustments will be within the ranges discussed below. That is the reactions will be conducted at mild conditions at all times.

While the auxiliary enzymes have been discussed as a mixture it is recognized that the enzymes may be added sequentially where the temperature, pH, and other conditions may be altered to increase the activity of each individual enzyme. Alternatively, an optimum pH and temperature can be determined for the enzyme mixture.

The enzymes are reacted with substrate under mild conditions that do not include extreme heat or acid treatment, as is currently utilized for biomass conversion using bioreactors. For example, enzymes can be incubated at about 25° C., about 30° C., about 35° C., about 37° C., about 40° C., about 45° C., about 50° C., or about 55° C. That is, they can be incubated from about 20° C. to about 70° C., in buffers of low to medium ionic strength, and neutral pH. By “medium ionic strength” is intended that the buffer has an ion concentration of about 200 millimolar (mM) or less for any single ion component. The pH may range from about pH 5, about pH 5.5, about pH 6, about pH 6.5, about pH 7, about pH 7.5, about pH 8.0, to about pH 8.5. Generally, the pH range will be from about pH 4.5 to about pH 9. Incubation of enzyme combinations under these conditions results in release or liberation of substantial amounts of the sugar from the lignocellulose. By substantial amount is intended at least 20%, 30%, 40%, 50%, 60%, 70%, 80%, 90%, 95% or more of available sugar.

A pretreatment step involving incubation with an enzyme or enzyme mixture can be utilized. The pretreatment step can be performed at many different temperatures but it is preferred that the pretreatment occur at the temperature best suited to the enzyme mix being tested, or the predicted enzyme optimum of the enzymes to be tested. The temperature of the pretreatment may range from about 10° C. to about 80° C., about 20° C. to about 80° C., about 30° C. to about 70° C., about 40° C. to about 60° C., about 37° C. to about 50° C., preferably about 37° C. to about 80° C., more preferably about 50° C. In the absence of data on the temperature optimum, it is preferable to perform the pretreatment reactions at 37° C. first, then at a higher temperature such as 50° C. The pH of the pretreatment mixture may range from about 2.0 to about 10.0, but is preferably about 3.0 to about 7.0, more preferably about 4.0 to about 6.0, even more preferably about 4.5 to about 5. Again, the pH may be adjusted to maximize enzyme activity and may be adjusted with the addition of the enzyme. Comparison of the results of the assay results from this test will allow one to modify the method to best suit the enzymes being tested.

The pretreatment reaction may occur from several minutes to several hours, such as from about 6 hours to about 120 hours, preferably about 6 hours to about 48 hours, more preferably about 6 to about 24 hours, most preferably for about 6 hours. The cellulase treatment may occur from several minutes to several hours, such as from about 6 hours to about 120 hours, preferably about 12 hours to about 72 hours, more preferably about 24 to 48 hours.

Biomass Substrate Definitions

By “substrate” or “biomass” is intended materials containing cellulose, hemicellulose, lignin, protein, and carbohydrates, such as starch and sugar. “Biomass” includes virgin biomass and or non-virgin biomass such as agricultural biomass, commercial organics, construction and demolition debris, municipal solid waste, waste paper and yard waste. Common forms of biomass include trees, shrubs and grasses, wheat, wheat straw, sugar cane bagasse, corn, corn husks, corn kernel including fiber from kernels, products and by-products from milling of grains such as corn (including wet milling and dry milling) as well as municipal solid waste, waste paper and yard waste. “Blended biomass” is any mixture or blend of virgin and non-virgin biomass, preferably having about 5-95% by weight non-virgin biomass. “Agricultural biomass” includes branches, bushes, canes, corn and corn husks, energy crops, forests, fruits, flowers, grains, grasses, herbaceous crops, leaves, bark, needles, logs, roots, saplings, short rotation woody corps, shrubs, switch grasses, trees, vegetables, vines, and hard and soft woods (not including woods with deleterious materials). In addition, agricultural biomass includes organic waste materials generated from agricultural processes including farming and forestry activities, specifically including forestry wood waste. Agricultural biomass may be any of the aforestated singularly or in any combination of mixture thereof.

Biomass high in starch, sugar, or protein such as corn, grains, fruits and vegetables are usually consumed as food. Conversely, biomass high in cellulose, hemicellulose and lignin are not readily digestible and are primarily utilized for wood and paper products, fuel, or are typically disposed. Generally, the substrate is of high lignocellulose content, including corn stover, rice straw, hay, sugarcane bagasse, and other agricultural biomass, switchgrass, forestry wastes, poplar wood chips, pine wood chips, sawdust, yard waste, and the like, including any combination of substrate.

Examples of Materials Typically Referred to as Biomass

| | | Residue from | |
|---|---|---|---|
| Non-Agricultural plant | Agricultural plant | Agricultural | Non-plant |
| material | material | processing | Material |
| Trees | Wheat straw | Corn Fiber | Refuse |
| Shrubs | Sugar cane bagasse | Residue from | Paper |
| | | corn processing | |
| Grasses | Rice Straw | | |
| Wood Chips | Switchgrass | | |
| Sawdust | Corn stover | | |
| Yard waste | Corn grain | | |
| Grass clippings | Corn fiber | | |
| Forestry wood waste | Vegetables | | |
| | Fruits | | |

By “liberate” or “hydrolysis” is intended the conversion of complex lignocellulosic substrates or biomass to simple sugars and oligosaccharides.

“Conversion” includes any biological, chemical and/or bio-chemical activity which produces ethanol or ethanol and byproducts from biomass and/or blended biomass. Such conversion includes hydrolysis, fermentation and simultaneous saccharification and fermentation (SSF) of such biomass and/or blended biomass. Preferably, conversion includes the use of fermentation materials and hydrolysis materials as defined herein.

“Corn stover” includes agricultural residue generated by harvest of corn plants. Stover is generated by harvest of corn grain from a field of corn; typically by a combine harvester. Corn stover includes corn stalks, husks, roots, corn grain, and miscellaneous material such as soil in varying proportions.

“Corn fiber” is a fraction of corn grain, typically resulting from wet milling, dry milling, or other corn grain processing. The corn fiber fraction contains the fiber portion of the harvested grain remaining after extraction of starch and oils. Corn fiber typically contains hemicellulose, cellulose, residual starch, protein and lignin.

“Ethanol” includes ethyl alcohol or mixtures of ethyl alcohol and water.

“Fermentation products” includes ethanol, citric acid, butanol and isopropanol as well as derivatives thereof.

Enzyme Nomenclature and Definitions

The nomenclature recommendations of the IUBMB are published in Enzyme Nomenclature 1992 [Academic Press, San Diego, Calif., ISBN 0-12-227164-5 (hardback), 0-12-227165-3 (paperback)] with Supplement 1 (1993), Supplement 2 (1994), Supplement 3 (1995), Supplement 4 (1997) and Supplement 5 (in Eur. J. Biochem. (1994) 223:1-5; Eur. J. Biochem. (1995) 232:1-6 ; Eur. J. Biochem. (1996) 237:1-5 ; Eur. J. Biochem. (1997) 250:1-6, and Eur. J. Biochem. (1999) 264:610-650; respectively). The classifications recommended by the IUBMB are widely recognized and followed in the art. Typically, enzymes are referred to in the art by the IUBMB enzyme classification, or EC number. Lists of enzymes in each class are updated frequently, and are published by IUBMB in print and on the internet.

Another source for enzyme nomenclature base on IUBMB classifications can be found in the ENZYME database. ENZYME is a repository of information relative to the nomenclature of enzymes. It is primarily based on the recommendations of the Nomenclature Committee of the International Union of Biochemistry and Molecular Biology (IUBMB) and it describes each type of characterized enzyme for which an EC (Enzyme Commission) number has been provided (Bairoch (2000) Nucleic Acids Res 28:304-305). The ENZYME database describes for each entry: the EC number, the recommended name, alternative names (if any), the catalytic activity, cofactors (if any), pointers to the SWISS-PROT protein sequence entrie(s) that correspond to the enzyme (if any), and pointers to human disease(s) associated with a deficiency of the enzyme (if any).

“Cellulase” includes both exohydrolases and endohydrolases that are capable of recognizing cellulose, or products resulting from cellulose breakdown, as substrates. Cellulase includes mixtures of enzymes that include endoglucanases, cellobiohydrolases, glucosidases, or any of these enzymes alone, or in combination with other activities. Organisms producing a cellulose-degrading activity often produce a plethora of enzymes with different substrate specificities. Thus, a strain identified as digesting cellulose may be described as having a cellulase, when in fact several enzyme types may contribute to the activity. For example, commercial preparations of ‘cellulase’ are often mixtures of several enzymes, such as endoglucanase, exoglucanase, and glucosidase activities.

Thus, “cellulase” includes mixtures of such enzymes, and includes commercial preparations capable of degrading cellulose, as well as culture supernatant or cell extracts exhibiting cellulose-degrading activity, or acting on the breakdown products of cellulose degradation, such as cellotriose or cellobiose.

“Cellobiohydrolase” or “1,4,-β-D-glucan cellobiohydrolase” or “cellulose 1,4-β-cellobiosidase” or “cellobiosidase” includes enzymes that hydrolyze 1,4-β-D-glucosidic linkages in cellulose and cellotetraose, releasing cellobiose from the reducing or non-reducing ends of the chains. Enzymes in group EC 3.2.1.91 include these enzymes.

“β-glucosidase” or “glucosidase” or “β-D-glucoside glucohydrolase” or “cellobiase” EC 3.2.1.21 includes enzymes that release glucose molecules as a product of their catalytic action. These enzymes recognize polymers of glucose, such as cellobiose (a dimer of glucose linked by β-1,4 bonds) or cellotriose (a trimer of glucose linked by β-1,4 bonds) as substrates. Typically they hydrolyze the terminal, non-reducing β-D-glucose, with release of β-D-glucose.

“Endoglucanase” or “1,4-β-D-glucan 4-glucanohydrolase” or “β-1,4, endocellulase” or “endocellulase”, or “cellulase” EC 3.2.1.4 includes enzymes that cleave polymers of glucose attached by β-1,4 linkages. Substrates acted on by these enzymes include cellulose, and modified cellulose substrates such as carboxymethyl cellulose, RBB-cellulose, and the like.

Cellulases include but are not limited to the following list of classes of enzymes.

| Name Used in | | | | |
|---|---|---|---|---|
| this | | EC | | |
| application | EC Name | Classification | Alternate Names | Reaction catalyzed |
| 1,4-β- | Cellulase | 3.2.1.4 | Endoglucanase; | Endohydrolysis of 1,4-β-D- |
| endoglucanase | | | Endo-1,4-β- | glucosidic linkages |
| | | | glucanase; | |
| | | | Carboxymethyl | |
| | | | cellulase; | |
| | | | β-1,4-endoglucanase; | |
| | | | 1,4-β-endoglucanase | |
| 1,3-β- | Endo-1,3(4)- | 3.2.1.6 | Endo-1,4-β- | Endohydrolysis of 1,3- or |
| endoglucanase | β-glucanase | | glucanase; | 1,4-linkages in β-D-glucans |
| | | | Endo-1,3-β- | when the reducing glucose |
| | | | glucanase; | residue is substituted at C-3 |
| | | | Laminarinase; | |
| | | | 1,3-β-endoglucanase | |
| β-glucosidase | β-glucosidase | 3.2.1.21 | Gentobiase; | Hydrolysis of terminal, |
| | | | Cellobiase; | non-reducing β-D-glucose |
| | | | Amygdalase | residues with release of β- |
| | | | | D-glucose |
| 1,3-1,4-β- | Licheninase | 3.2.1.73 | Lichenase; | Hydrolysis of 1,4-β-D- |
| endoglucanase | | | β-glucanase; | glycosidic linkages in β-D- |
| | | | Endo-β-1,3-1,4 | glucans containing 1,3- and |
| | | | glucanase; | 1,4-bonds |
| | | | 1,3-1,4-β-D-glucan; | |
| | | | 4-glucanohydrolase; | |
| | | | Mixed linkage β- | |
| | | | glucanase; | |
| | | | 1,3-1,4-β- | |
| | | | endoglucanase | |
| 1,3-1,4-β- | Glucan 1,4-β- | 3.2.1.74 | Exo-1,4-β- | Hydrolysis of 1,4-linkages |
| exoglucanase | glucosidase | | glucosidase; | in 1,4-β-D-glucans so as to |
| | | | 1,3-1,4-β- | remove successive glucose |
| | | | exoglucanase | units |
| Cellobiohydrolase | Cellulose 1,4- | 3.2.1.91 | Exoglucanase; | Hydrolysis of 1,4-β-D- |
| | β- | | Exocellobiohydrolase; | glucosidic linkages in |
| | cellobiosidase | | 1,4-β- | cellulose and cellotetraose, |
| | | | cellobiohydrolase; | releasing cellobiose from |
| | | | Cellobiohydrolase | the reducing or non- |
| | | | | reducing ends of the chains |

“Xylanase” or “Hemicellulase” includes both exohydrolytic and endohydrolytic enzymes that are capable of recognizing and hydrolyzing hemicellulose, or products resulting from hemicellulose breakdown, as substrates. In monocots, where heteroxylans are the principle constituent of hemicellulose, a combination of endo-1,4-β-xylanase (EC 3.2.1.8) and β-D-xylosidase (EC 3.2.1.37) may be used to break down hemicellulose to xylose. Additional debranching enzymes are capable of hydrolyzing other sugar components (arabinose, galactose, mannose) that are located at branch points in the hemicellulose structure. Additional enzymes are capable of hydrolyzing bonds formed between hemicellulosic sugars (notably arabinose) and lignin.

“Endoxylanase” or “1,4-β-endoxylanase” or “1,4-β-D-xylan xylanohydrolase” or (EC 3.2.1.8) include enzymes that hydrolyze xylose polymers attached by β-1,4 linkages. Endoxylanases can be used to hydrolyze the hemicellulose component of lignocellulose as well as purified xylan substrates.

“Exoxylanase” or “β-xylosidase” or “xylan 1,4-β-xylosidase” or “1,4-β-D-xylan xylohydrolase” or “xylobiase” or “exo-1,4-β-xylosidase” (EC 3.2.1.37) includes enzymes that hydrolyze successive D-xylose residues from the non-reducing terminus of xylan polymers.

“Arabinoxylanase” or “glucuronoarabinoxylan endo-1,4-β-xylanase” or “feraxan endoxylanase” includes enzymes that hydrolyze β-1,4 xylosyl linkages in some xylan substrates.

Xylanases include but are not limited to the following group of enzymes.

| Name Used in | | EC | Alternate | |
|---|---|---|---|---|
| this application | EC Name | Classification | Names | Reaction catalyzed |
| 1,4-β- | Endo-1,4-β- | 3.2.1.8 | 1,4-β-D-xylan; | Endohydrolysis of 1,4-β-D- |
| endoxylanase | xylanase | | xylanohydrolase; | xylosidic linkages in xylans |
| | | | 1,4-β-endoxylanase | |
| 1,3-β- | Xylan endo- | 3.2.1.32 | Xylanase; | Random hydrolysis of 1,3- |
| endoxylanase | 1,3-β- | | Endo-1,3-β- | β-D-xylosidic linkages in |
| | xylosidase | | xylanase; | 1,3-β-D-xylans |
| | | | 1,3-β-endoxylanase | |
| β-xylosidase | Xylan 1,4-β- | 3.2.1.37 | β-xylosidase; | Hydrolysis of 1,4-β-D- |
| | xylosidase | | 1,4-β-D-xylan | xylans removing successive |
| | | | xylohydrolase; | D-xylose residues from the |
| | | | Xylobiase; | non-reducing termini |
| | | | Exo-1,4-β- | |
| | | | xylosidase | |
| Exo-1,3-β- | Xylan 1,3-β- | 3.2.1.72 | Exo-1,3-β- | Hydrolysis of successive |
| xylosidase | xylosidase | | xylosidase | xylose residues from the |
| | | | | non-reducing termini of 1,3- |
| | | | | β-D-xylans |
| Arabinoxylanase | Glucuronoarabinoxylan | 3.2.1.136 | Feraxan | Endohydrolysis of 1,4-β-D- |
| | endo-1,4-β- | | endoxylanase; | xylosyl links in some |
| | xylanase | | Arabinoxylanase | gluconoarabinoxylans |

“Ligninases” includes enzymes that can hydrolyze or break down the structure of lignin polymers. Enzymes that can break down lignin include lignin peroxidases, manganese peroxidases, laccases and feruloyl esterases, and other enzymes described in the art known to depolymerize or otherwise break lignin polymers. Also included are enzymes capable of hydrolyzing bonds formed between hemicellulosic sugars (notably arabinose) and lignin.

Ligninases include but are not limited to the following group of enzymes.

| Name Used | | | |
|---|---|---|---|
| in this | EC | | Reaction |
| application | Classification | Alternate Names | catalyzed |
| Lignin | 1.11.1 | none | Oxidative |
| peroxidase | | | degradation |
| | | | of lignin |
| Manganese | 1.11.1.13 | Mn-dependent | Oxidative |
| peroxidase | | peroxidase | degradation |
| | | | of lignin |
| Laccase | 1.10.3.2 | Urishiol oxidase | Oxidative |
| | | | degradation |
| | | | of lignin |
| Feruloyl | 3.1.1.73 | Ferulic acid esterase; | Hydrolyzes |
| esterase | | Hydroxycinnamoyl | bonds between |
| | | esterase; Cinnamoyl | arabinose |
| | | ester hydrolase | and lignin |

“Amylase” or “alpha glucosidase” includes enzymes that hydrolyze 1,4-α-glucosidic linkages in oligosaccharides and polysaccharides. Many amylases are characterized under the following EC listings:

| Name Used in | EC | | |
|---|---|---|---|
| this application | Classification | Alternate Names | Reaction catalyzed |
| Alpha-amylase | 3.2.1.1 | 1,4-alpha-D-glucan | Hydrolysis of 1,4-alpha-glucosidic |
| | | glucanohydrolase; | linkages |
| | | Glycogenase | |
| Beta-amylase | 3.2.1.2 | 1,4-alpha-D-glucan | Hydrolysis of terminal 1,4-linked |
| | | maltohydrolase; | alpha-D-glucose residues |
| | | saccharogen amylase | |
| | | Glycogenase | |
| Glucan 1,4-alpha- | 3.2.1.3 | Glucoamylase; 1,4- | Hydrolysis of terminal 1,4-linked |
| glucosidase | | alpha-D-glucan | alpha-D-glucose residues |
| | | glucohydrolase; | |
| | | Amyloglucosidase; | |
| | | Gamma-amylase; | |
| | | Lysosomal alpha- | |
| | | glucosidase; Exo-1,4- | |
| | | alpha-glucosidase | |
| Alphaglucosidase | 3.2.1.20 | Maltase; | Hydrolysis of terminal, non-reducing |
| | | Glucoinvertase; | 1,4-linked D-glucose |
| | | Glucosidosucrase; | |
| | | Maltase- | |
| | | glucoamylase; | |
| | | Lysosomal alpha- | |
| | | glucosidase; Acid | |
| | | maltase | |
| Glucan 1,4-alpha- | 3.2.1.60 | Exo- | Hydrolysis of 1,4-alpha-D-glucosidic |
| maltotetrahydrolase | | maltotetraohydrolase; | linkages |
| | | G4-amylase; | |
| | | Maltotetraose-forming | |
| | | amylase | |
| Isoamylase | 3.2.1.68 | Debranching enzyme | Hydrolysis of alpha-(1,6)-D- |
| | | | glucosidic linkages in glycogen, |
| | | | amylopectin and their beta-limits |
| | | | dextrins |
| Glucan-1,4-alpha- | 3.2.1.98 | Exomaltohexaohydrolase; | Hydrolysis of 1,4-alpha-D-glucosidic |
| maltohexaosidase | | Maltohexaose- | linkages |
| | | producing amylase; | |
| | | G6-amylase | |
| Glucan-1,4-alpha- | 3.2.1.133 | Maltogenic alpha- | Hydrolysis of(1 →4)-alpha-D- |
| maltohydrolase | | amylase | glucosidic linkages in |
| | | | polysaccharides |
| Cyclomaltodextrin | 2.4.1.19 | Cyclodextrin- | Degrades starch to cyclodextrins by |
| glucanotransferase | | glycosyltransferase; | formation of a 1,4-alpha-D- |
| | | Bacillus macerans | glucosidic bond |
| | | amylase; Cyclodextrin | |
| | | glucanotransferase | |
| Oligosaccharide | 2.4.1.161 | Amylase III | Transfer the non-reducing terminal |
| 4-alpha-D- | | | alpha-D-glucose residue from a 1,4- |
| glucosyl- | | | alpha-D-glucan to the 4-position of |
| transferase | | | an alpha-D-glucan |

“Protease” includes enzymes that hydrolyze peptide bonds (peptidases), as well as enzymes that hydrolyze bonds between peptides and other moieties, such as sugars (glycopeptidases). Many proteases are characterized under EC 3.4, and are incorporated herein by reference. Some specific types of proteases include, cysteine proteases including pepsin, papain and serine proteases including chymotrypsins, carboxypeptidases and metalloendopeptidases. The SWISS-PROT Protein Knowledgebase (maintained by the Swiss Institute of Bioinformatics (SIB),Geneva, Switzerland and the European Bioinformatics Institute (EBI),Hinxton, United Kingdom) classifies proteases or peptidases into the following classes.

Serine-type peptidases

| Family | Representative enzyme |
|---|---|
| S1 | Chymotrypsin/trypsin |
| S2 | Alpha-Lytic endopeptidase |
| S2 | Glutamyl endopeptidase (V8) (Staphylococcus) |
| S2 | Protease Do (htrA) (Escherichia) |
| S3 | Togavirin |
| S5 | Lysyl endopeptidase |
| S6 | IgA-specific serine endopeptidase |
| S7 | Flavivirin |
| S29 | Hepatitis C virus NS3 endopeptidase |
| S30 | Tobacco etch virus 35 kDa endopeptidase |
| S31 | Cattle diarrhea virus p80 endopeptidase |
| S32 | Equine arteritis virus putative endopeptidase |
| S35 | Apple stem grooving virus serine endopeptidase |
| S43 | Porin D2 |
| S45 | Penicillin amidohydrolase |
| S8 | Subtilases |
| S8 | Subtilisin |
| S8 | Kexin |
| S8 | Tripeptidyl-peptidase II |
| S53 | Pseudomonapepsin |
| S9 | Prolyl oligopeptidase |
| S9 | Dipeptidyl-peptidase IV |
| S9 | Acylaminoacyl-peptidase |
| S10 | Carboxypeptidase C |
| S15 | Lactococcus X-Pro dipeptidyl-peptidase |
| S28 | Lysosomal Pro-X carboxypeptidase |
| S33 | Prolyl aminopeptidase |
| S11 | D-Ala-D-Ala peptidase family 1 (E. coli dacA) |
| S12 | D-Ala-D-Ala peptidase family 2 (Strept. R61) |
| S13 | D-Ala-D-Ala peptidase family 3 (E. coli dacB) |
| S24 | LexA repressor |
| S26 | Bacterial leader peptidase I |
| S27 | Eukaryote signal peptidase |
| S21 | Assemblin (Herpesviruses protease) |
| S14 | ClpP endopeptidase (Clp) |
| S49 | Endopeptidase IV (sppA) (E. coli) |
| S41 | Tail-specific protease (prc) (E. coli) |
| S51 | Dipeptidase E (E. coli) |
| S16 | Endopeptidase La (Lon) |
| S19 | Coccidiodes endopeptidase |
| S54 | Rhomboid |

Threonine-type peptidases

| Family | Representative enzyme |
|---|---|
| T1 | Multicatalytic endopeptidase (Proteasome) |

Cysteine-type peptidases

| Family | Representative enzyme |
|---|---|
| C1 | Papain |
| C2 | Calpain |
| C10 | Streptopain |
| C3 | Picornain |
| C4 | Potyviruses NI-a (49 kDa) endopeptidase |
| C5 | Adenovirus endopeptidase |
| C18 | Hepatitis C virus endopeptidase 2 |
| C24 | RHDV/FC protease P3C |
| C6 | Potyviruses helper-component (HC) proteinase |
| C7 | Chestnut blight virus p29 endopeptidase |
| C8 | Chestnut blight virus p48 endopeptidase |
| C9 | Togaviruses nsP2 endopeptidase |
| C11 | Clostripain |
| C12 | Ubiquitin C-terminal hydrolase family 1 |
| C13 | Hemoglobinase |
| C14 | Caspases (ICE) |
| C15 | Pyroglutamyl-peptidase I |
| C16 | Mouse hepatitis virus endopeptidase |
| C19 | Ubiquitin C-terminal hydrolase family 2 |
| C21 | Turnip yellow mosaic virus endopeptidase |
| C25 | Gingipain R |
| C26 | Gamma-glutamyl hydrolase |
| C37 | Southampton virus endopeptidase |
| C40 | Dipeptidyl-peptidase VI (Bacillus) |
| C48 | SUMO protease |
| C52 | CAAX prenyl protease 2 |

Aspartic-type peptidases

| Family | Representative enzyme |
|---|---|
| A1 | Pepsin |
| A2 | Retropepsin |
| A3 | Cauliflower mosaic virus peptidase |
| A9 | Spumaretrovirus endopeptidase |
| A11 | Drosophila transposon copia endopeptidase |
| A6 | Nodaviruses endopeptidase |
| A8 | Bacterial leader peptidase II |
| A24 | Type IV-prepilin leader peptidase |
| A26 | Omptin |
| A4 | Scytalidopepsin |
| A5 | Thermopsin |

Metallopeptidases

| Family | Representative enzyme |
|---|---|
| M1 | Membrane alanyl aminopeptidase |
| M2 | Peptidyl-dipeptidase A |
| M3 | Thimet oligopeptidase |
| M4 | Thermolysin |
| M5 | Mycolysin |
| M6 | Immune inhibitor A (Bacillus) |
| M7 | Streptomyces small neutral protease |
| M8 | Leishmanolysin |
| M9 | Microbial collagenase |
| M10 | Matrixin |
| M10 | Serralysin |
| M10 | Fragilysin |
| M11 | Autolysin (Chlamydomonas) |
| M12 | Astacin |
| M12 | Reprolysin |
| M13 | Neprilysin |
| M26 | IgA-specific metalloendopeptidase |
| M27 | Tentoxilysin |
| M30 | Staphylococcus neutral protease |
| M32 | Carboxypeptidase Taq |
| M34 | Anthrax lethal factor |
| M35 | Deuterolysin |
| M36 | Aspergillus elastinolytic metalloendopeptidase |
| M37 | Lysostaphin |
| M41 | Cell division protein ftsH (E. coli) |
| M46 | Pregnancy-associated plasma protein-A |
| M48 | CAAX prenyl protease |
| M49 | Dipeptidyl-peptidase III |

Others without HEXXH motifs

| Family | Representative enzyme |
|---|---|
| M14 | Carboxypeptidase A |
| M14 | Carboxypeptidase H |
| M15 | Zinc D-Ala-D-Ala carboxypeptidase |
| M45 | Enterococcus D-Ala-D-Ala dipeptidase |
| M16 | Pitrilysin |
| M16 | Mitochondrial processing peptidase |
| M44 | Vaccinia virus-type metalloendopeptidase |
| M17 | Leucyl aminopeptidase |
| M24 | Methionyl aminopeptidase, type 1 |
| M24 | X-Pro dipeptidase |
| M24 | Methionyl aminopeptidase, type 2 |
| M18 | Yeast aminopeptidase I |
| M20 | Glutamate carboxypeptidase |
| M20 | Gly-X carboxypeptidase |
| M25 | X-His dipeptidase |
| M28 | Vibrio leucyl aminopeptidase |
| M28 | Aminopeptidase Y |
| M28 | Aminopeptidase iap (E. coli) |
| M40 | Sulfolobus carboxypeptidase |
| M42 | Glutamyl aminopeptidase (Lactococcus) |
| M38 | E. coli beta-aspartyl peptidase |
| M22 | O-Sialoglycoprotein endopeptidase |
| M52 | Hydrogenases maturation peptidase |
| M50 | SREBP site 2 protease |
| M50 | Sporulation factor IVB (B. subtilis) |
| M19 | Membrane dipeptidase |
| M23 | Beta-Lytic endopeptidase |
| M29 | Thermophilic aminopeptidase |

Peptidases of unknown catalytic mechanism

| Family | Representative enzyme |
|---|---|
| U3 | Spore endopeptidase gpr (Bacillus) |
| U4 | Sporulation sigmaE factor processing peptidase (Bacillus) |
| U6 | Murein endopeptidase (mepA) (E. coli) |
| U8 | Bacteriophage murein endopeptidase |
| U9 | Prohead endopeptidase (phage T4) |
| U22 | Drosophila transposon 297 endopeptidase |
| U24 | Maize transposon bs1 endopeptidase |
| U26 | Enterococcus D-Ala-D-Ala carboxypeptidase |
| U29 | Encephalomyelitis virus endopeptidase 2A |
| U30 | Commelina yellow mottle virus proteinase |
| U31 | Human coronavirus protease |
| U32 | Porphyromonas collagenase |
| U33 | Rice tungro bacilliform virus endopeptidase |
| U34 | Lactococcal dipeptidase A |

“Lipidase” includes enzymes that hydrolyze lipids, fatty acids, and acylglycerides, including phospoglycerides, lipoproteins, diacylglycerols, and the like. In plants, lipids are used as structural components to limit water loss and pathogen infection. These lipids includes waxes derived from fatty acids, as well as cutin and suberin. Many lipases are characterized under the following EC listings:

| Name Used | | | |
|---|---|---|---|
| in this | EC | | |
| application | Classification | Alternate Names | Reaction catalyzed |
| Triacylglycerol | 3.1.1.3 | Lipase; Triglyceride | Triacylglycerol + H₂O [symbol] |
| lipase | | lipase; Tributyrase | diacylglycerol + a fatty acid anion |
| Phospholipase | 3.1.1.4 | Phosphatidylcholine 2- | Phosphatidylcholine + H₂O [symbol] 1- |
| A2 | | acylhydrolase; | acylglycerophosphocholine + a fatty |
| | | Lecithinase A; | acid anion |
| | | Phosphatidase; | |
| | | Phosphatidolipase | |
| Lysophospholipase | 3.1.1.5 | Lecithinase B; | 2-lysophosphatidylcholine + H₂O [symbol] |
| | | Lysolecithinase; | glycerophosphocholine + a fatty acid |
| | | Phospholipase B | anion |
| Acylglycerol | 3.1.1.23 | Monoacylglycerol | Hydrolyzes glycerol monoesters of |
| lipase | | lipase | long-chain fatty acids |
| Galactolipase | 3.1.1.26 | None | 1,2-diacyl-3-beta-D-galactosyl-sn- |
| | | | glycerol + 2 H₂O [symbol] 3-beta-D- |
| | | | galactosyl-sn-glycerol + 2 fatty acid |
| | | | anion |
| Phospholipase | 3.1.1.32 | None | Phosphatidylcholine + H₂O [symbol] 2- |
| A1 | | | acylglycerophosphocholine + a fatty |
| | | | acid anion |
| Dihydrocoumarin | 3.1.1.35 | None | Dihydrocoumarin + H₂O [symbol] |
| lipase | | | melilotate |
| 2-acetyl-1- | 3.1.1.47 | 1-alkyl-2- | 2-acetyl-1-alkyl-sn-glycero-3- |
| alkylglycerophosphocholine | | acetylglycerophosphocholine | phosphocholine + H₂O [symbol] 1-alkyl-sn- |
| esterase | | esterase; Platelet- | glycero-3-phosphocholine + acetate |
| | | activing factor | |
| | | acetylhydrolase; PAF | |
| | | acetylhydrolase; PAF | |
| | | 2-acylhydrolase; LDL- | |
| | | associated | |
| | | phospholipase A2; | |
| | | LDL-PLA(2) | |
| Phosphatidylinositol | 3.1.1.52 | Phosphatidylinositol | 1-phosphatidyl-1D-myoinositol + H₂O |
| deacylase | | phospholipase A2 | [symbol] 1-acylglycerophosphoinositol + a fatty |
| | | | acid anion |
| Cutinase | 3.1.1.74 | None | Cutis + H₂O [symbol] cutis monomers |
| Phospholipase C | 3.1.4.3 | Lipophosphodiesterase | A phosphatidylcholine + H₂O [symbol] 1,2 |
| | | I; Lecithinase C; | diacylglycerol + choline phosphate |
| | | Clostridium welchii | |
| | | alpha-toxin, | |
| | | Clostridium | |
| | | oedematiens beta- and | |
| | | gamma toxins | |
| Phospholipase D | 3.1.4.4 | Lipophosphodiesterase | A phosphatidylcholine + H₂O [symbol] |
| | | II; Lecithinase D; | choline + a phosphatidate |
| | | Choline phosphatase | |
| 1-phosphatidylinositol | 3.1.4.10 | Monophosphatidylinositol | 1-phosphatidyl-1D-myoinositol [symbol] |
| phosphodiesterase | | phosphodiesterase; | 1D-mylinositol 1,2-cyclic phosphate + diacylglycerol |
| | | phosphatidylinositol | |
| | | phospholipase C | |
| Alkylglycero- | 3.1.4.39 | Lysophospholipase D | 1-alkyl-sn-glycero-3- |
| phosphoethanolamine | | | phosphoethanolamine + H₂O [symbol] 1- |
| phosphodiesterase | | | alkyl-sn-glycerol 3-phosphate + ethanolamine |

“Glucuronidase” includes enzymes that catalyze the hydrolysis of β-glucuronoside to yield an alcohol. Many glucoronidases are characterized under the following EC listings:

| Name Used | | | |
|---|---|---|---|
| in this | EC | | |
| application | Classification | Alternate Names | Reaction catalyzed |
| Beta- | 3.2.1.31 | None | A beta-D-glucuronosidase + H₂O [symbol] |
| glucuronidase | | | an alcohol + D-glucuronate |
| Hyalurono- | 3.2.1.36 | Hyaluronidase | Hydrolysis of 1,3-linkages between |
| glucuronidase | | | beta-D-glucuronate and N-acetyl-D- |
| | | | glucosamine |
| Glucuronosyl- | 3.2.1.56 | None | 3-D-glucuronosyl-N(2)-6-disulfo- |
| disulfoglucosamine | | | beta-D-glucosamine + H₂O [symbol] N(2)- |
| glucuronidase | | | 6-disulfo-D-glucosamine + D- |
| | | | glucuronate |
| Glycyrrhizinate | 3.2.1.128 | None | Glycyrrhizinate + H₂O [symbol] 1,2-beta- |
| beta- | | | D-glucuronosyl-D-glucuronate + glycyrrhetinate |
| glucuronidase | | | |
| Alpha- | 3.2.1.139 | Alpha-glucuronidase | An alpha-D-glucuronosidase + H₂O |
| glucosiduronase | | | [symbol] an alcohol + D-glururonate |

Methods for degrading substrate using enzyme mixtures to liberate sugars

In one aspect of the invention, the enzymes act on lignocellulosic substrates or plant biomass, serving as the feedstock, and convert this complex substrate to simple sugars and oligosaccharides for the production of ethanol or other useful products. Another aspect of the invention includes methods that utilize mixtures of enzymes that act synergistically with other enzymes or physical treatments such as temperature and pH to convert the lignocellulosic plant biomass to sugars and oligosaccharides. Enzyme combinations or physical treatments can be administered concomitantly or sequentially. The enzymes can be produced either exogenously in microorganisms, yeasts, fungi, bacteria or plants, then isolated and added to the lignocellulosic feedstock. Alternatively, the enzymes are produced, but not isolated, and crude cell mass fermentation broth, or plant material (such as corn stover), and the like are added to the feedstock. Alternatively, the crude cell mass or enzyme production medium or plant material may be treated to prevent further microbial growth (for example, by heating or addition of antimicrobial agents), then added to the feedstock. These crude enzyme mixtures may include the organism producing the enzyme. Alternatively, the enzyme may be produced in a fermentation that uses feedstock (such as corn stover) to provide nutrition to an organism that produces an enzyme(s). In this manner, plants that produce the enzymes may serve as the lignocellulosic feedstock and be added into lignocellulosic feedstock.

Sugars released from biomass can be converted to useful fermentation products including, but not limited to, amino acids, vitamins, pharmaceuticals, animal feed supplements, specialty chemicals, chemical feedstocks, plastics, and ethanol, including fuel ethanol.

The enzyme mixtures can be expressed in microorganisms, yeasts, fungi or plants. Methods for the expression of the enzymes are known in the art. See, for example, Sambrook et al. (1989) Molecular Cloning: A Laboratory Manual (2d ed., Cold Spring Harbor Laboratory Press, Plainview, N.Y.); Ausubel et al., eds. (1995) Current Protocols in Molecular Biology (Greene Publishing and Wiley-Interscience, New York); U.S. Pat. Nos: 5,563,055; 4,945,050; 5,886,244; 5,736,369; 5,981,835; and others known in the art, all of which are herein incorporated by reference. In one aspect of this invention the enzymes are produced in transgenic plants. In this method the plants express some or all of the auxiliary enzyme(s) utilized for conversion of biomass to simple sugars or oligosaccharides.

Methods to Identify Enzymes and Strains Producing Enzymes for Use in the Method

In another aspect of the invention, methods to identify enzymes capable of acting as auxiliary enzymes to degrade lignocellulosic biomass are provided. To identify novel enzymes with the ability to facilitate degradation of lignocellulosic material, such as corn stover, one can utilize the assays described herein.

First, one identifies and clones a set of genes likely to act as auxiliary enzymes. One may generate such a pool of genes by sorting a database of known lignocellulose-degrading enzymes, for example, and then identifying genes to clone. The choice of which enzyme-producing genes to clone can depend on several factors. One may wish to identify particular genes whose products are known or suspected to have particular properties. These properties include, for example, activity at high or low pH values, activity in high salt concentration, high temperatures, the ability to encode proteins of a certain size or amino acid composition, having activity on certain substrates, or being members of certain classes of proteins. Next, the desired set of genes are amplified using methods known in the art, for example PCR (from strains containing these genes). Alternatively, one may design and synthesize the gene(s) by annealing and extending synthetic oliogonucleotides. Methods for such gene synthesis are known in the art. Subsequently, the resulting DNA is cloned into an expression vector in a manner such that the predicted proteins can be expressed in a cell (such as an E. coli cell).

Second, one expresses protein from these genes in, for example, E. coli, and prepares extracts that contain the activity to test. One may achieve this by generating lysates from these cells, harvesting supernatants containing the activity, or by purifying the activity, for example by column chromatography.

Third, one tests the extracts prepared in this way using assays known in the art, and identifies clones that produce activity in the assays used. In contrast to current methods, complex mixtures of polymeric carbohydrates and lignin, or actual lignocellulose are used as the substrate attacked by biomass conversion enzymes. One assay that may be used to measure the release of sugars and oligosaccharides from these complex substrates is the dinitrosalicylic acid assay (DNS). In this assay, the lignocellulosic material such as corn stover is incubated with enzymes(s) for various times and the released reducing sugars measured. This assay uses any complex lignocellulosic material, including corn stover, sawdust, woodchips, and the like.

In one aspect of this invention the lignocellulosic material is pretreated with a auxiliary enzyme mix. This mix is composed of enzymes from (1) commercial suppliers; (2) cloned genes expressing enzymes; (3) complex broth (such as that resulting from growth of a microbial strain in media, wherein the strains secrete proteins and enzymes into the media; (4) cell lysates of strains grown as in (3); and, (5) plant material expressing enzymes capable of degrading lignocellulose.

Following pretreatment, the lignocellulosic material may be treated with a cellulose-degrading enzyme such as the enzyme mixture from T. reesei. Aliquots of the mixtures may be taken at various time points before and after addition of the assay constituents, and the release of sugars may be measured by a DNS assay.

In another aspect of this invention, the treatment with auxiliary enzymes and a cellulase occurs in the same reaction vessel. In this aspect, one performs the steps as above, except that the cellulase treatment and auxiliary enzyme treatment are combined.

Using these assays one can assess the ability of the tested auxiliary enzyme mix to produce sugars from lignocellulose. Furthermore, one can measure the conversion of lignocellulose to sugars and oligosaccharides by various enzymes, enzyme combinations or physical treatments.

The use of complex lignocellulosic substrates such as corn stover and corn fiber in assays such as those described in this invention allows testing and measurement of synergies between enzyme classes that degrade different components of lignocellulose (for example cellulose, hemicellulose, and or lignin).

Methods to Identify Synergistic Enzyme Combinations

Also provided are methods to identify the optimum ratios and compositions of enzymes with which to degrade each lignocellulosic material. These methods entail tests to identify the optimum enzyme composition and ratios for efficient conversion of any lignocellulosic substrate to its constituent sugars.

By using lignocellulosic substrates such as corn stover, rice straw, hay, sugarcane bagasse, and other agricultural biomass, switchgrass, forestry wastes, poplar wood chips, pine wood chips, sawdust, yard waste and the like, in tests as described, and measuring the amount of sugar or oligosaccharide released, the synergy between the classes of enzymes that convert different components of lignocellulose can be measured. For example, the ratio of an endoxylanase and a cellulase (or preparation comprised of a mixture of several cellulases and other enzymes) required to give high activity on corn stover can be measured. Subsequently, the ratio of such enzymes required for efficient degradation of a different lignocellulosic substrate (e.g. corn fiber) can be determined by the methods provided herein.

The following examples are offered by way of illustration and not by way of limitation.

### EXPERIMENTAL

### EXAMPLE 1.

### High Throughput Quantitation of Release of Reducing Sugars and Oligosaccharides from Corn Stover

A small amount of dried corn stover (approximately 30 g) is ground in a Waring blender for 5 minute intervals to produce a coarse powder mixture. Processing the stover in this fashion increases uniformity of the particle size and reduces the heterogeneity of the sample due to heterogeneity in individual corn stalks and plant residue. In this example, 0.2 g of ground stover material is placed in a 50 ml conical tube for each assay sample. The stover is washed with 15 ml of 100 mM sodium acetate buffer (pH 6.0) to remove any unbound sugars. This slurry is vortexed for 30 seconds, centrifuged for 5 minutes at 4000 rpm, and the supernatant is removed by pipetting.

The stover sample is resuspended in 10 ml of the enzyme solution or sterile filtered supernatant to be assayed. The mixture is then incubated at the desired temperature in an air shaker at 250-300 rpm. At appropriate time points the stover suspensions are removed from the shaker and centrifuged for 5 minutes at 4000 rpm. A small volume of supernatant (approximately 300 μl) is removed from the tube and transferred to a 1.5 ml microcentrifuge tube, and assayed by a DNS assay.

### EXAMPLE 2.

### Pretreatment of Corn Stover With Xylanase Prior to Cellulase-Mediated Degadation to Enhance Release of Soluble Sugars

Samples of corn stover (0.2 mg per tube; washed and prepared in buffer as described above) were incubated in a pretreatment reaction for 6 hours at 37° C. with either 0, 10 or 100 units of xylanase from Trichoderma viride. At the end of pretreatment, each sample was treated with 100 units of cellulase from Trichoderma reesei and incubated for 18 hours at 37° C. Liberation of soluble sugars was monitored by measuring the amount of reducing sugar using a DNS method. Table 1 shows the release of soluble sugars over time (as detected by DNS absorbance at 540 nm). Each time point in Table 1 reflects the average of 4 independent measurements. The pretreatment step was observed to substantially increase the conversion of stover to soluble sugars following addition of cellulase.

TABLE 1

| Xylanase Pretreatment | Reducing Sugar Release |
|---|---|
| (activity units) | (A₅₄₀) |
| 0 | 2.57 |
| 10 | 3.84 |
| 100 | 4.73 |

### EXAMPLE 3.

### Co-Treatment of Corn Stover With Purified Cellulase and Xylanase Enzymes to Enhance Release of Soluble Sugars

Samples of corn stover (0.2 mg per tube; washed and prepared in buffer as described above) were incubated for 6 hours at 37° C. with either 10 units, 100 units or 500 units of xylanase from T. viride. Simultaneously, samples containing 100 units of cellulase from T. reesei were co-treated with either 0 units, 10 units, 100 units or 500 units of xylanase from T. viride for 6 hours at 37° C. Liberation of soluble sugars was quantified by removing 300 μl aliquots and measuring the amount of reducing sugar using a DNS method. Table 2 shows the release of soluble sugars (as detected by DNS absorbance at 540 nm). Each time point in Table 2 reflects the average of four independent measurements. The co-treatment was observed to liberate substantially more sugar than either enzyme alone, or the sum of the activities of either enzyme.

TABLE 2

| Cellulase | Xylanase | Reducing Sugar Release |
|---|---|---|
| (activity units) | (activity units) | (A₅₄₀) |
| 0 | 10 | 0.1 |
| 0 | 100 | 0.3 |
| 0 | 500 | 0.6 |
| 100 | 0 | 2.1 |
| 100 | 10 | 2.4 |
| 100 | 100 | 3.4 |
| 100 | 500 | 3.9 |

### EXAMPLE 4.

### Co-Treatment of Stover With Cellulase and Xylanase Liberates Substantial Amounts of Sugars

Samples of corn stover (0.2 mg per tube; washed and prepared in buffer as described above) were co-treated with cellulase enzyme (500 units, T. reesei) and xylanase (500 units, T. viride) at 0, 24 and 48 hours. Untreated controls were also prepared. Following 24 and 120 hours of incubation at 37° C., the release of soluble sugars was detected by DNS absorbance at 540 nm. Each data point in Table 3 reflects the average of four independent measurements.

TABLE 3

| Time | Stover Hydrolysis, | Stover Hydrolysis, |
|---|---|---|
| (hours) | No enzymes | cellulase + xylanase |
| 0 | 0.3% | 0.3% |
| 24 | 0.4% | 32.1% |
| 120 | 0.8% | 37.6% |

### EXAMPLE 5:

### Identification of Microbial Strains Capable of Degrading Corn Stover

Microorganisms are grown in culture flasks (typically a 50 mL cultures in 250 mL baffled flask) in a rich growth medium (such as Luria broth). Mesophilic strains are typically grown for 48 hrs at 30° C., and thermophilic strains are typically grown for 18 hours at 65° C. Following the growth of individual strains, the cells are centrifuged at 5000 rpm for 10 minutes to clarify the supernatant, and the supernatant is further sterilized by passage through syringe filter units or vacuum filter sterilization units. The sterilized culture filtrate is further concentrated using a concentration unit. One method of concentration of proteins in supernatant makes use of spin filter concentration units (such as Microcon/Centricon/Centriprep units from Millipore with 3000 molecular weight cutoff), but other concentration methods would also be appropriate. This sterilized culture supernatant (or concentrated culture filtrate) is assayed for the ability to degrade corn stover.

Clarified supernatants are mixed with stover substrate in the following manner: Approximately 30 g of corn stover is ground in a Waring blender for 2×5 minute intervals on the “High” setting. For each extract to be screened, 4 mls of concentrated supernatant is added to 0.1 g of ground stover and 1 ml of 100 mM sodium acetate pH 5.0 (as a buffer). Each tube is then placed in a rack in an incubator-shaker and incubated overnight at 50° C. with shaking (16-20 hours). Individual samples are centrifuged briefly to separate the starting biomass substrate from any soluble reducing sugars that have been released from the substrate into the supernatant. Individual tubes are tested for release of reducing sugars from stover using a DNS assay.

### EXAMPLE 6.

### Identification of Strains that Produce Auxiliary Enzymes Acting on Corn Stover

Strains producing auxiliary enzymes may not result in degradation of corn stover as described above. To identify strains that produce auxiliary enzymes, one may test for strains that produce enzymes that facilitate subsequent cellulase degradation. Culture filtrates prepared and concentrated as in Example 6 are incubated with stover for various times (as in example 6). Following the incubation of stover with secreted proteins, the tubes are boiled for 20 minutes to destroy enzyme and protease activities. After boiling, tubes are cooled to 50° C., and 100 units of cellulase (Trichoderma reesei) is added to each tube. The tubes are incubated at 50° C. for 16-20 hours. Following this incubation, reducing sugars are quantified by a DNS assay.

More than 100 microbial strains were screened as described in this method. Strains were grown and sterilized, and concentrated culture supernatant was prepared from the grown cultures. These filtrates were assayed for the ability to degrade corn stover as described above, and the amount released reducing sugars quantified. The assay of 12 strains that do not degrade stover yield average DNS value at A540 nm of 0.113±0.23. Several strains exhibited an ability to liberate sugar that was significantly better than controls, and significantly better than strains that show basal level activity (greater than 3 standard deviations above the average). These activities are shown in Table 4.

Thus, the methods of the invention are useful in identifying strains useful in degradation of plant biomass, including corn stover.

TABLE 4

| Strain Number | Reducing sugar release (A₅₄₀) |
|---|---|
| ATX3661 | 1.004 |
| ATX6024 | 0.450 |
| ATX1410 | 0.395 |
| ATX6027 | 0.242 |
| ATX5975 | 0.226 |
| ATX4221 | 0.207 |

### EXAMPLE 7.

### Identification of Enzymes With Ability to Degrade Corn Fiber and Distiller's Dried Grains

The assays described herein can be adapted for use with other lignocellulose substrates. In this example, corn fiber is adapted to the assay, and enzymes are tested for the ability to degrade corn fiber and distiller's dried grains.

Samples of corn fiber or distiller's dried grains (1.0 g per tube; washed and prepared in buffer as described above) were treated with cellulase enzyme (500 units, T. reesei) or xylanase (500 units, T. viride). Untreated controls were also prepared alongside. Following 0 and 24 hours of incubation at 37° C., the release of soluble sugars was detected by DNS absorbance at 540 nm. Each data point in Table 5 reflects the average of four independent measurements.

TABLE 5

| | | | Distiller's dried | |
|---|---|---|---|---|
| | Corn Fiber | | grains | |
| | Hydrolysis, | | Hydrolysis, | Distiller's dried |
| | 500 units | Corn Fiber | 500 units | grains |
| Time | cellulase + | Hydrolysis, | cellulase + | Hydrolysis, |
| (hours) | xylanase | No enzymes | xylanase | No enzymes |
| 0 | 2.2 | 2.2 | 2.0 | 1.9 |
| 24 | 14.6 | 2.2 | 8.8 | 2.0 |

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method for degrading lignocellulose to sugars, said method comprising contacting said lignocellulose with at least one auxiliary enzyme and at least one cellulase for a time sufficient to liberate said sugars, wherein at least 20% of said sugars are liberated in the absence of high temperature and pressure.

2. The method of claim 1, wherein said auxiliary enzyme is added as a crude or a semi-purified enzyme mixture.

3. The method claim 1, wherein said auxiliary enzyme is produced by culturing at least one organism on a substrate to produce said enzyme.

4. The method of claim 3, wherein said organism is selected from the group consisting of a bacterium, a fungus, and a yeast.

5. The method of claim 1, wherein said auxiliary enzyme is produced in a plant cell.

6. The method of claim 1, wherein said lignocellulose is contacted with more than one auxiliary enzyme.

7. The method of claim 1, wherein said auxiliary enzyme is a xylanase.

8. The method of claim 1, wherein said lignocellulose is selected from the group consisting of corn stover, corn fiber, Distiller's dried grains from corn, rice straw, hay, sugarcane bagasse, barley, malt and other agricultural biomass, switchgrass, forestry wastes, poplar wood chips, pine wood chips, sawdust, and yard waste.

9. The method of claim 8, wherein said lignocellulose comprises corn stover.

10. The method of claim 8, wherein said lignocellulose comprises corn fiber.

11. The method of claim 8, wherein said lignocellulose comprises Distiller's dried grains.

12. The method of claim 1, wherein said auxiliary enzyme is incubated with said lignocellulose prior to the addition of said cellulase.

13. A method for degrading a stover to sugars, said method comprising contacting said stover with a xylanase and a cellulase for a time sufficient to liberate said sugars, wherein at least 20% of said sugars are liberated in the absence of high temperature and pressure.

14. The method of 13, wherein said xylanase is an endoxylanase.

15. The method of 14, wherein said cellulase is an endocellulase.

16. The method of 14, wherein said cellulase is an exocellulase.
